(19) United States
(12) Patent Application Publication
(10) Pub. No.: US 2025/0143712 A1
(43) Pub. Date: May 8, 2025

(54) EXTRAVASCULAR SECURING ELEMENT FOR PERCUTANEOUS DEVICE ENABLED LEFT ATRIAL APPENDAGE OCCLUSION

(71) Applicant: BOSTON SCIENTIFIC SCIMED, INC., MAPLE GROVE, MN (US)

(72) Inventors: Jerry Wang, Edina, MN (US); Joshua Tillman, New Brighton, MN (US); Graham Krumpelmann, Stillwater, MN (US); Philip Andrew Litecky, Forest Lake, MN (US)

(73) Assignee: BOSTON SCIENTIFIC SCIMED, INC., MAPLE GROVE, MN (US)

(21) Appl. No.: 18/934,492

(22) Filed: Nov. 1, 2024

Related U.S. Application Data
(60) Provisional application No. 63/595,840, filed on Nov. 3, 2023.

(51) Int. Cl. A61B 17/12

(52) U.S. Cl. CPC A61B 17/12122; A61B 17/12031

(57) ABSTRACT

An implant for left atrial appendage closure includes an expandable framework having a proximal end coupled to a collar, a shaft disposed within the collar and axially moveable relative to the collar, and a lock coupled to the shaft. The expandable framework is configured to expand from a collapsed delivery configuration to an expanded deployed configuration. The lock includes an engagement member configured to move between a constrained configuration and a radially expanded configuration, where the engagement member is biased in the radially expanded configuration.

### CROSS-REFERENCE TO RELATED APPLICATIONS

This application claims the benefit of priority of U.S. Provisional Application No. 63/595,840 filed Nov. 3, 2023, the entire disclosure of which is hereby incorporated by reference.

### TECHNICAL FIELD

The disclosure pertains to medical devices and more particularly to devices for left atrial appendage occlusion, and methods for using such medical devices.

### BACKGROUND

A wide variety of medical devices have been developed for medical use including, for example, medical devices utilized to occlude regions of the body. These medical devices may be used in a variety of body regions including an aneurysm in a vessel and the left atrial appendage (LAA). In patients suffering from atrial fibrillation, the LAA may not properly contract or empty, causing stagnant blood to pool within its interior, which can lead to the undesirable formation of thrombi within the LAA.

Thrombi forming in the LAA may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the LAA. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example implant for left atrial appendage closure includes an expandable framework having a proximal end coupled to a collar, the expandable framework configured to expand from a collapsed delivery configuration to an expanded deployed configuration, a shaft disposed within the collar and axially moveable relative to the collar, and a lock coupled to the shaft, the lock including an engagement member configured to move between a constrained configuration and a radially expanded configuration, wherein the engagement member is biased in the radially expanded configuration.

Alternatively or additionally to the embodiment above, the collar holds the engagement member in the constrained configuration, and axial movement of the shaft within the collar moves the collar off the engagement member, allowing the engagement member to move into the radially expanded configuration.

Alternatively or additionally to any of the embodiments above, the lock includes a plurality of arms, each arm having a first end coupled to the shaft and a second free end, the second free end configured to move between the constrained configuration and the radially expanded configuration.

Alternatively or additionally to any of the embodiments above, the second free end of each arm extends in a proximal direction in the radially expanded configuration.

Alternatively or additionally to any of the embodiments above, the collar holds the second free end of each arm in the constrained configuration, and axial movement of the shaft within the collar releases the second free end of each of the plurality of arms.

Alternatively or additionally to any of the embodiments above, the expandable framework is biased in the expanded deployed configuration.

Alternatively or additionally to any of the embodiments above, the expandable framework includes a plurality of struts each having a proximal end, a middle portion, and distal end, wherein the proximal ends are coupled to the collar, the distal ends are coupled together, and the middle portions are moveable between the collapsed delivery configuration and the expanded deployed configuration, wherein the plurality of struts are biased in the expanded deployed configuration.

Alternatively or additionally to any of the embodiments above, at least the middle portion of each strut has a plurality of projections extending laterally from the strut.

Alternatively or additionally to any of the embodiments above, the implant further includes a delivery rod removably coupled to a proximal end of the shaft, the delivery rod configured to rotate the shaft and move the shaft axially relative to the expandable framework.

Alternatively or additionally to any of the embodiments above, the shaft includes a fastener and a nut disposed around the fastener, the nut having at least one opening in a sidewall thereof, wherein the collar has at least one aperture through a sidewall thereof, the implant further comprising at least one pin extending through the aperture in the collar and the opening in the nut.

Alternatively or additionally to any of the embodiments above, the fastener threadingly engages the nut such that rotation of the delivery rod in a first direction causes the fastener to move in a first axial direction through the nut and the collar, and rotation of the delivery rod in a second direction opposite the first direction causes the fastener to move in a second axial direction through the nut and the collar.

Alternatively or additionally to any of the embodiments above, the first end of each of the plurality of arms is coupled adjacent a proximal end of the shaft, and each arm extends distally with the collar holding the second free end of each arm in the constrained configuration.

Alternatively or additionally to any of the embodiments above, movement of the shaft distally through the collar moves the second free ends of the plurality of arms out from under the collar, allowing the arms to expand into the radially expanded configuration.

Alternatively or additionally to any of the embodiments above, the second free end of each arm bends radially away from a longitudinal axis of the shaft, and then bends proximally as the shaft moves distally through the collar.

Alternatively or additionally to any of the embodiments above, the first end of each of the plurality of arms is coupled adjacent a distal end of the shaft, and each arm extends proximally with the collar holding the second free end of each arm in the constrained configuration.

Alternatively or additionally to any of the embodiments above, movement of the shaft distally through the collar moves the collar proximally off the second free ends of the plurality of arms, allowing the second free ends of the arms to bend radially away from a longitudinal axis of the shaft.

Another example implant for left atrial appendage closure includes an expandable framework having a proximal end coupled to a collar, the expandable framework configured to expand from a collapsed delivery configuration to a radially expanded configuration, a shaft disposed within the collar and axially moveable relative to the collar, and a lock coupled to the shaft, the lock including a plurality of arms each having a first end coupled to the shaft and a second free end configured to move between a constrained configuration when positioned under the collar and a radially expanded configuration when released from the collar, the plurality of arms biased in the radially expanded configuration, wherein the shaft is configured to rotate in a first direction causing the shaft to move axially through the collar to move the plurality of arms into the radially expanded configuration.

Alternatively or additionally to any of the embodiments above, the first end of each of the plurality of arms is coupled adjacent a proximal end of the shaft, and each arm extends distally with the collar holding the second free end of each arm in the constrained configuration.

Alternatively or additionally to any of the embodiments above, the first end of each of the plurality of arms is coupled adjacent a distal end of the shaft, and each arm extends proximally with the collar holding the second free end of each arm in the constrained configuration.

An example method of closing a left atrial appendage includes the steps of inserting an implant into the left atrial appendage, the implant including an expandable framework having a proximal end coupled to a collar, the expandable framework configured to expand from a collapsed delivery configuration to an expanded deployed configuration, at least a portion of the expandable framework having a plurality of projections extending laterally therefrom, a shaft disposed within the collar and axially moveable relative to the collar, a lock coupled to the shaft, the lock including an engagement member configured to move between a constrained configuration and a radially expanded configuration, wherein when in the radially expanded configuration, the engagement member is configured to engage tissue at a proximal end of the shaft, and a delivery rod removably coupled to a proximal end of the shaft. The method further includes inserting a torque shaft over the delivery rod and into engagement with the expandable framework, rotating the torque shaft in a first direction to rotate the expandable framework in the first direction and engage the plurality of projections with an inner surface of the left atrial appendage, rotating the delivery rod relative to the torque shaft in a second direction opposite the first direction to rotate the shaft in the second direction, thereby axially moving the shaft through the collar to move the engagement member into the radially expanded configuration, wherein the engagement member engages tissue of the left atrial appendage at a proximal region of the shaft, and removing the torque shaft and the delivery rod, leaving the proximal end of the shaft within tissue of the left atrial appendage.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a perspective view of an example occlusion device and delivery rod;

FIG. 2 is a side cross-sectional view of the occlusion device and delivery rod of FIG. 1;

FIGS. 3A-3D are perspective views of the occlusion device and delivery rod of FIG. 1 during deployment;

FIG. 4 is a side view of the occlusion device of FIG. 1 deployed in the LAA;

FIG. 5 is a perspective view of another example occlusion device and delivery rod;

FIG. 6 is a side cross-sectional view of the occlusion device and delivery rod of FIG. 5;

FIGS. 7A-7E are perspective views of the occlusion device and delivery rod of FIG. 5 during deployment;

FIG. 8 is a side view of the occlusion device of FIG. 5 deployed in the LAA; and

FIG. 9 is a side cross-sectional view of an alternate rod and shaft connection.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

After implantation of a left atrial appendage (LAA) occlusion device, there remains an ongoing desire to eliminate leaks around the device and avoid device elements in the circulatory system to reduce device-related thrombus (DRT) and requirement for post-op oral anticoagulation therapy. The embodiments discussed below address these needs by having the securing element of the LAA occlusion device within the sealed/occluded section of the LAA hidden from the endovascular/circulatory system where DRT can develop.

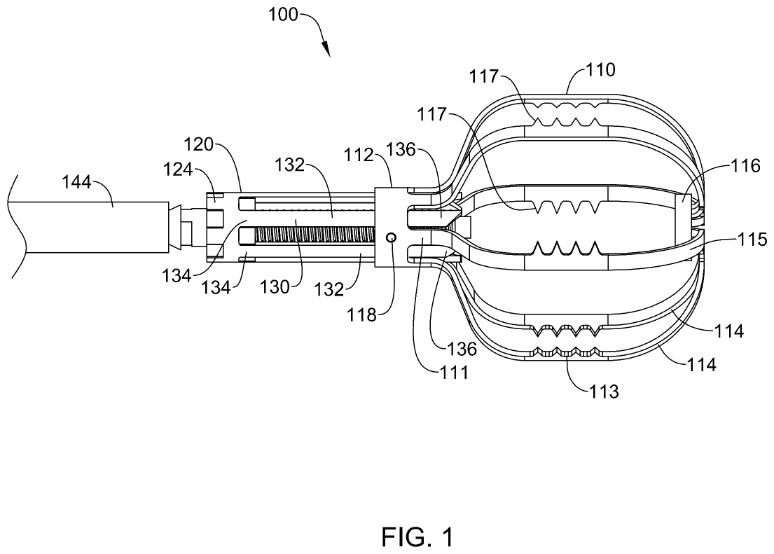

FIG. 1 illustrates an occlusive implant 100 and delivery sleeve 144 for occluding the LAA and maintaining all structure within the LAA such that no structure is in fluid contact with the circulatory system. It should be noted that in any given figure, some features of the occlusive implant 100 may not be shown, or may be shown schematically, for simplicity. Additional details regarding some of the components of the occlusive implant 100 may be illustrated in other figures in greater detail. The occlusive implant 100 may include an expandable framework 110, a shaft 120, and a lock 130 coupled to the shaft. The expandable framework 110 may have a proximal end coupled to a collar 112. The shaft 120 may be reversibly coupled to the delivery sleeve 144 for delivery and actuation. The expandable framework 110 may be configured to expand from a collapsed delivery configuration to an expanded deployed configuration. The expandable framework 110 is shown in the expanded deployed configuration in FIG. 1. The expandable framework may be biased in the expanded deployed configuration, and may be made of a shape memory material such as nitinol. The expandable framework 110 may be self-expandable.

In some embodiments, the expandable framework 110 may include a plurality of struts 114 each having a proximal end 111, a middle portion 113, and distal end 115. The proximal ends 111 may be fixed to the collar 112. In some embodiments, the struts 114 and the collar 112 may be formed as a single monolithic structure. The distal ends 115 of the struts may be coupled to one another or they may be coupled to another structure. In the embodiment shown in FIG. 1, the distal ends 115 of the struts 114 are coupled to a retainer 116. The distal ends 115 may be bent back proximally and coupled to the retainer disposed within an interior space defined by the struts, as shown in FIG. 1. In other embodiments, the distal ends 115 may extend distally with the retainer 116 positioned outside the interior space. The middle portions 113 of each strut 114 are moveable between the collapsed delivery configuration and the expanded deployed configuration, and may be biased in the expanded deployed configuration. The implant 100 may be delivered through a sheath configured to hold the struts 114 in the collapsed configuration. In the collapsed delivery configuration, the expandable framework may be in a substantially cylindrical configuration, with the struts 114 extending substantially linearly from the collar 112. Withdrawal of the sheath or advancement of the implant 100 distally out of the sheath allows the struts 114 to radially expand into the configuration shown in FIG. 1. The expandable framework 110 or at least the struts 114 may be made of a shape memory material, heat set in the expanded configuration.

At least the middle portion 113 of each strut may have a plurality of projections 117 extending laterally from the strut. The projections 117 may be a plurality of teeth 117 as shown in FIG. 1. The teeth 117 may be disposed only on one side of the struts 114 when looking axially down the expandable framework from the delivery sleeve 144 to the distal ends 115 of the struts. This configuration allows the struts to engage and grip the inner wall of the LAA when the expandable framework 110 is rotated in a first direction to twist the LAA with the implant, while allowing the expandable framework to slide relative to the inner wall when rotated in a second direction opposite the first direction. In the embodiment illustrated in FIG. 1, the teeth 117 extend only on the left side of the struts 114 when looking from the proximal ends 111 to the distal ends 115 of the struts, which causes the teeth 117 to grip or become embedded in the inner wall of the LAA when the implant 100 is rotated counter-clockwise. In other embodiments, the projections 117 may extend from both sides of the struts, or radially outward from the outer surface (facing the LAA) of the struts, or any combination thereof.

The shaft 120 and the lock 130 may be disposed within the collar 112 and may be axially moveable relative to the collar. The lock 130 is configured to lock the implant 100 to the LAA and hold the LAA closed. The lock 130 may include an engagement member configured to move between a constrained delivery configuration and a radially expanded configuration, with the engagement member biased in the radially expanded configuration. The collar 112 may hold the engagement member in the constrained configuration, and axial movement of the shaft through the collar 112 moves the collar off the engagement member, allowing the engagement member to move into the radially expanded configuration.

In the embodiment shown in FIGS. 1-4, the engagement member of the lock 130 includes a plurality of arms 132, each arm 132 having a first end 134 coupled to the shaft adjacent the proximal end 124 of the shaft 120. Each arm 132 extends distally from the first end 134 to a second free end 136, the second free end configured to move between the constrained configuration and the radially expanded configuration. The collar 112 may hold the second free ends 136 against the shaft 120 in the constrained configuration, as shown in FIG. 1, and axial movement of the shaft within the collar releases the second free end of each of the plurality of arms, as will be discussed below.

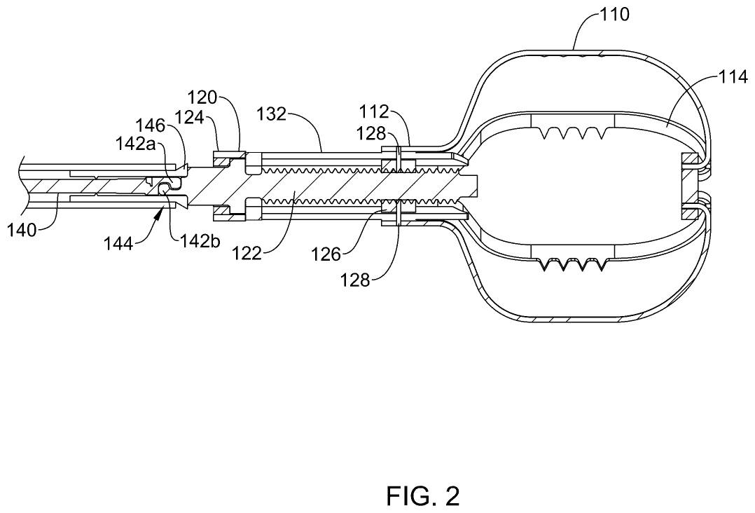

The cross-sectional view in FIG. 2 illustrates the elements of the delivery system, which may include a delivery rod 140 removably coupled to the proximal end of the shaft 120. The delivery rod 140 may be configured to rotate the shaft and expandable framework together, and also to move the shaft axially relative to the expandable framework. The distal end of the rod 140 may include a first coupler 142a and the proximal end 124 of the shaft 120 may include a second coupler 142b, which together form a two-piece coupling arrangement 142a, 142b. A removable cover 146 may be slidingly disposed over at least the two-piece coupling arrangement 142a, 142b and a portion of the delivery rod 140. The cover 146 may be fixed to the delivery sleeve 144, and the cover 146 may engage the rod 140 and first coupler 142a with a friction fit. During delivery, the sleeve 144 and cover 146 are disposed over the two-piece coupling arrangement 142a, 142b, as shown in FIG. 2. The sleeve 144 and cover 146 allow for rotation and axial movement of the sleeve 144 and rod 140 to be translated to rotation and axial movement of the occlusive implant 100. The two-piece coupling arrangement 142a, 142b may be configured to be released by lateral movement of the first coupler 142a relative to the second coupler 142b when the cover 146 has been withdrawn proximally, as discussed in more detail below. The covered coupling arrangement allows the rod 140 to rotate and drive the shaft 120 in both a counter-clockwise and a clockwise direction without becoming disengaged from the shaft.

The cross-sectional view in FIG. 2 also illustrates the elements of the shaft 120, including a fastener 122 and a nut 126 disposed around the fastener. The fastener 122 may be threaded and threadingly engaged with the nut 126. The nut 126 may have at least one opening in a sidewall thereof, and the collar 112 may have at least one aperture 118 through a sidewall thereof, with the opening in the nut 126 and the aperture in the collar 112 aligned such that at least one pin 128 may extend through one aperture in the collar and one opening in the nut to secure the collar 112 to the nut126. In the embodiment shown in FIG. 2, the nut 126 has two openings on opposite sides, the collar 112 has two apertures on opposite sides, and two pins 128 extend through the aligned openings and apertures to secure the collar to the nut. The fastener 122 threadingly engages the nut 126 such that rotation of the rod 140 in a first direction, such as counter clockwise, causes the fastener 122 to be withdrawn proximally through the nut 126 and collar 112, and rotation of the rod 140 in a second direction, such as clockwise, causes the fastener 122 to advance distally through the nut 126 and the collar 112 into the interior space defined by the expandable framework 110.

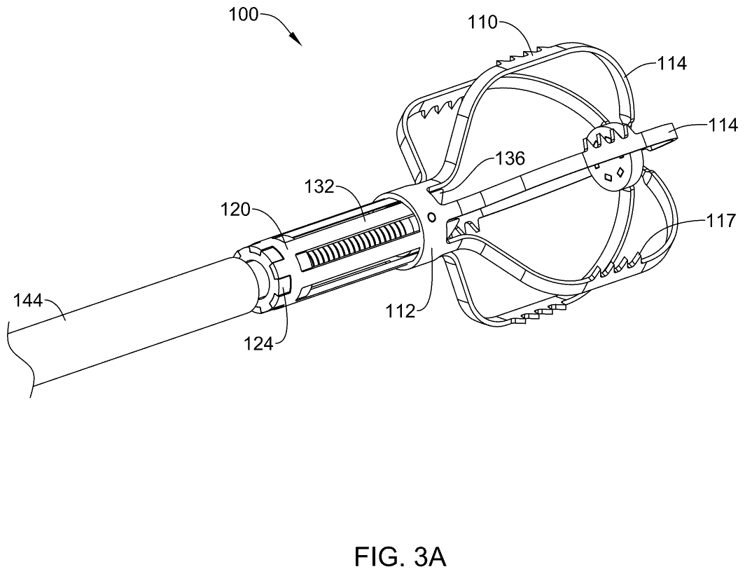

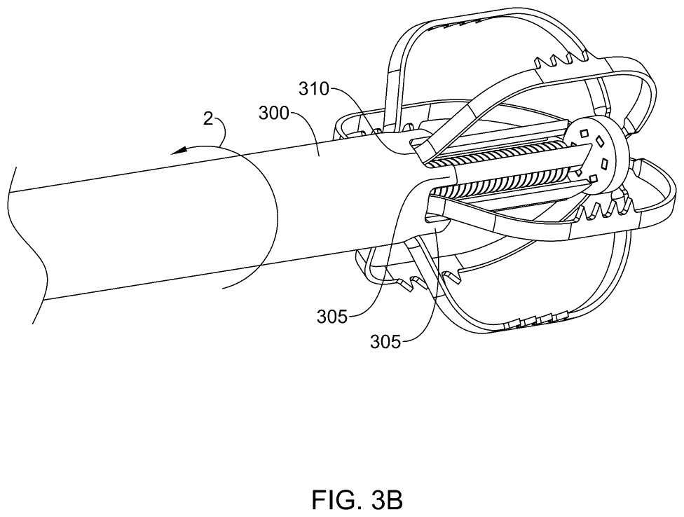

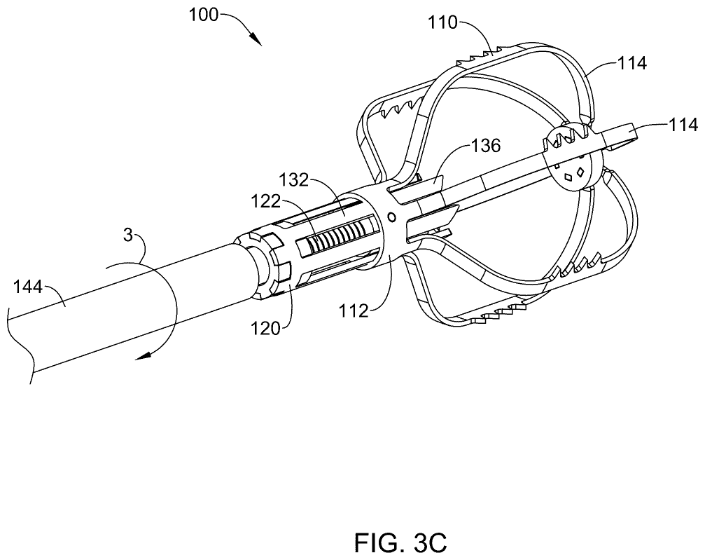

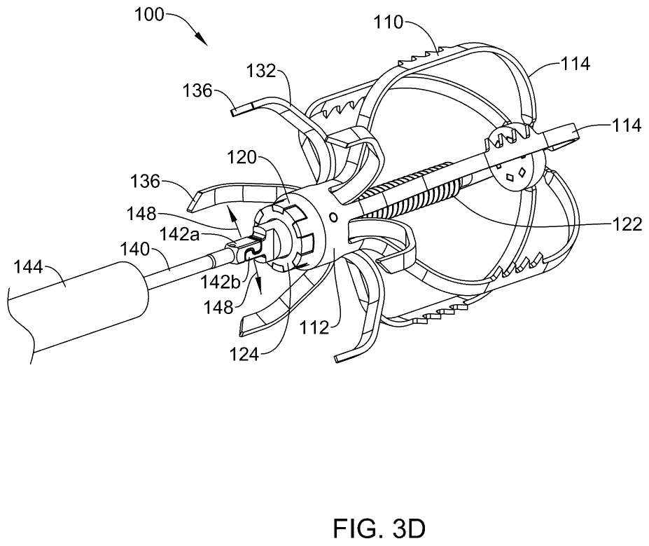

FIGS. 3A-3D illustrate the deployment of the lock arms 132. The implant 100 may be delivered into the LAA in a constrained configuration in a delivery sheath in which the struts 114 are substantially straight (not shown). When the delivery sheath is withdrawn proximally, the expandable framework 110 automatically expands radially into the configuration shown in FIG. 3A. In the delivery configuration and once the struts 114 have expanded, the collar 112 is positioned over the distal end of shaft 120, with the collar 112 over the free ends 136 of the arms 132, holding the arms 132 in their axial, constrained configuration, as shown in FIG. 3A.

Once the implant 100 has been delivered to the LAA and the expandable framework 110 has radially expanded with at least the middle portion 113 of the struts 114 in contact with the inner walls of the LAA, a torque shaft 300 may be advanced over the rod 140 and shaft 120. As shown in FIG. 3B, the distal end of the torque shaft 300 may have a plurality of axially extending fingers 305 with recesses 310 between adjacent fingers, where the fingers 305 are configured to be positioned between the struts 114 and the recesses 310 engage the proximal ends of the struts 114. With the torque shaft 300 thus engaged with the expandable framework 110, rotation of the torque shaft 300 in the first direction (counter-clockwise in this example), indicated by arrow 2, rotates the expandable framework 110 in the first direction, causing the projections 117 on the expandable framework 110 to grip or become embedded in the inner wall of the LAA, twisting the LAA around the implant 100 and closing off the opening of the LAA around the torque shaft 300. Once the implant 100 has been rotated and the LAA has twisted round the implant, the rod 140 and sleeve 144 may be rotated in the second direction opposite the first direction (clockwise in this example), indicated by arrow 3, which causes the shaft 120, including fastener 122, to move distally through the collar 112, as shown in FIG. 3C. The torque shaft 300 may remain engaged with the struts 114 to hold the LAA twisted around the implant 100, however the torque shaft 300 is not rotated clockwise with the rod 140 and shaft 120. The torque shaft 300 may be held stationary while the rod 140 is rotated within it. The torque shaft 300 is not shown in FIGS. 3C and 3D in order to illustrate the details of the shaft 120 and collar 112 during subsequent steps.

As the shaft 120 moves distally through the collar 112, the second free ends 136 of the plurality of arms move out from under the collar, allowing the arms to expand into the radially expanded configuration, as shown in FIG. 3D. As the arms 132 are moved distally out from under the collar, the second free end 136 of each arm bends radially away from a longitudinal axis of the shaft, and then bends proximally as the shaft moves distally through the collar, such that the free second ends 136 of the arms 132 extend in a proximal direction. When the shaft has moved distally into the interior space defined by the expandable framework 110, the collar 112 may be disposed over the first end of the arms, adjacent the proximal end 124 of the shaft. As shown in the progression from FIG. 3C to 3D, the free end 136 of each arm moves through 180 degrees to become embedded in the twisted tissue at what was the opening of the LAA. The free ends 136 of the arms 132 may prevent the tissue from untwisting, thereby holding the implant within the LAA and maintain the twist of tissue that completely closes off the LAA. In the embodiment shown in the figures, each arm 132 moves through a space between adjacent struts 114.

Once the free ends 136 of the arms 132 have become implanted in the twisted tissue at what was the opening of the LAA, the torque shaft 300, the sleeve 144, and attached cover 146 may be withdrawn proximally relative to the rod 140, uncovering the coupling arrangement 142a, 142b, which is then configured to release the rod 140 from the proximal end of shaft 120. The rod 140 with its first coupler 142a may be moved laterally relative to the second coupler 142b, indicated by arrows 148, thereby uncoupling the first coupler 142a from the second coupler 142b, as shown in FIG. 3D. For example, the coupling arrangement 142a, 142b may be a train latch type structure.

The initial twisting of the expandable framework 110 within the LAA with the projections 117 embedded in the inner wall of the LAA may cause the tissue at the neck of the LAA to become twisted around the second coupler 142b such that once the rod and first coupler 142a are removed, the entirety of the implant 100 including the proximal end of the shaft 120 and the second coupler 142b, is located within the extravascular space or tissue of the LAA, and no structure of the implant system extends into the endovascular space within the heart.

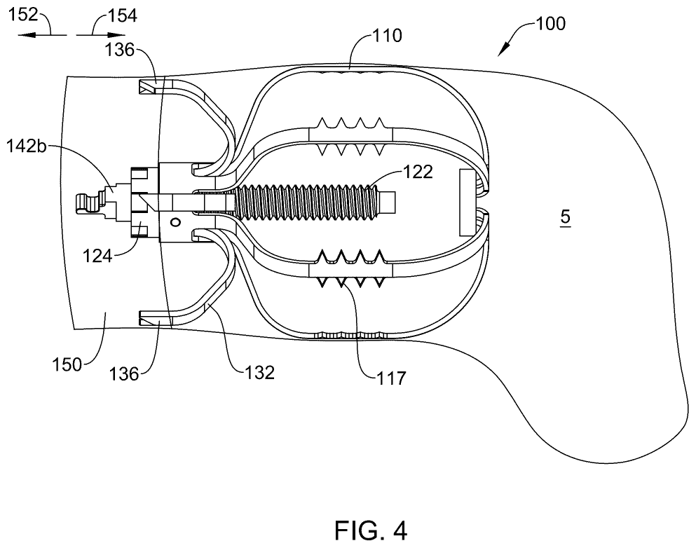

FIG. 4 shows the implant 100 deployed in the LAA 5. The projections 117 have engaged the inner wall of the LAA 5 and the implant was rotated in the first direction with the torque shaft 300, twisting the LAA and folding tissue at the neck of the LAA around the proximal end 124 of the shaft and the second coupler 142b. Rotation of the rod in the second direction caused deployment of the arms 132 of the lock, with the second free ends 136 of the arms 132 at least partially embedded in the tissue 150 at the neck of the LAA. The free ends 136 of arms 132 being embedded within the twisted tissue 150 at the neck of the LAA may prevent the tissue from untwisting. The lock thus provides a secure closure of the LAA without any structure in contact with the endovascular space 152 in the heart, eliminating device-related thrombus. In some embodiments, 0.01 inch to 0.1 inch of the second free ends 136 of the arms may be embedded within the tissue of the LAA. The shaft may have a length of 0.25 inch to 1.0 inch. Once the lock is deployed, the torque shaft 300 may be removed and the rod 140 and first coupler 142a may be uncoupled from the shaft, leaving the entire implant 100, including the second coupler 142b completely within the extravascular space 154 of the LAA, with no implant structure extending into the endovascular space 152 of the heart.

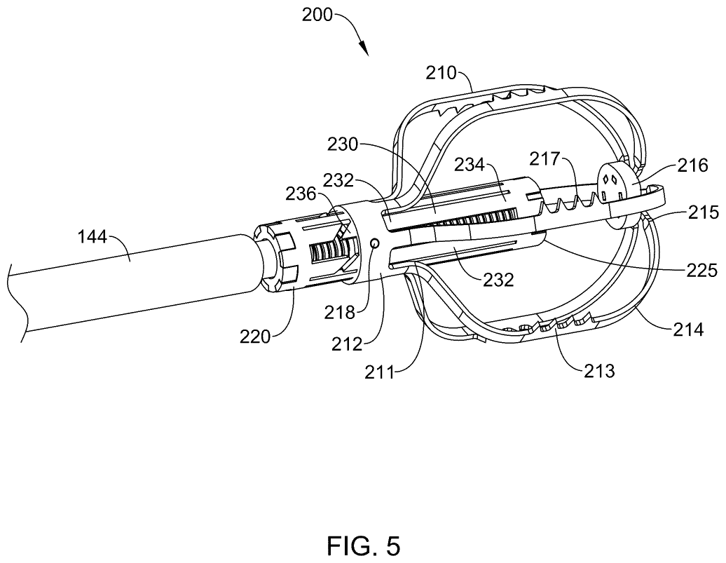

FIGS. 5-8 illustrate another embodiment of LAA occlusive implant 200 with a different lock 230. It should be noted that in any given figure, some features of the occlusive implant 200 may not be shown, or may be shown schematically, for simplicity. Additional details regarding some of the components of the occlusive implant 200 may be illustrated in other figures in greater detail. The occlusive implant 200 may include an expandable framework 210, a shaft 220, and a lock 230 coupled to the shaft. The expandable framework 210 may have a proximal end coupled to a collar 212. The shaft 220 may be reversibly coupled to the sleeve 144 for delivery. The expandable framework 210 may be configured to expand from a collapsed delivery configuration to an expanded deployed configuration. The expandable framework 210 is shown in the expanded deployed configuration in FIG. 5. The expandable framework may be biased in the expanded deployed configuration, and may be made of a shape memory material such as nitinol. The expandable framework 210 may be self-expandable.

The expandable framework 210 may include a plurality of struts 214 each having a proximal end 211, a middle portion 213, and distal end 215. The proximal ends 211 may be coupled to the collar 212. In some embodiments, the struts 214 and the collar 212 may be formed as a single monolithic structure. The distal ends 215 of the struts may be coupled to one another or they may be coupled to another structure. In the embodiment shown in FIG. 5, the distal ends 215 of the struts 214 are coupled to a retainer 216. The distal ends 215 may be bent back proximally and coupled to the retainer disposed within an interior space defined by the struts, as shown in FIG. 5. In other embodiments, the distal ends 215 may extend distally with the retainer 216 positioned outside the interior space. The middle portions 213 of each strut 214 are moveable between the collapsed delivery configuration and the expanded deployed configuration, and may be biased in the expanded deployed configuration. The implant 200 may be delivered through a sheath configured to hold the struts 214 in the collapsed configuration. In the collapsed delivery configuration, the expandable framework may be in a substantially cylindrical configuration, with the struts 214 extending substantially linearly from the collar 212. Withdrawal of the sheath or advancement of the implant 200 distally out of the sheath allows the struts 214 to radially expand into the configuration shown in FIG. 5. The expandable framework 210 or at least the struts 214 may be made of a shape memory material, heat set in the expanded configuration.

At least the middle portion 213 of each strut may have a plurality of projections 217 extending laterally from the strut. The projections 217 may be a plurality of teeth 217 as shown in FIG. 5. The teeth 217 may only be disposed on one side of the struts 214 when looking axially down the expandable framework from the sleeve 144 to the distal ends 215 of the struts. This configuration allows the struts to engage and grip the inner wall of the LAA when the expandable framework 210 is rotated in a first direction to twist the LAA with the implant, while allowing the expandable framework to slide relative to the inner wall when rotated in a second direction opposite the first direction. In the example illustrated in FIG. 5, the teeth 217 extend only on the left side of the struts 214 when looking from the proximal ends 211 to the distal ends 215 of the struts, which causes the teeth 217 to grip or become embedded in the inner wall of the LAA when the implant 200 is rotated counter clockwise. In other embodiments, the projections 217 may extend from both sides of the struts, or radially outward from the outer surface (facing the LAA) of the struts, or any combination thereof.

The shaft 220 and the lock 230 may be disposed within the collar 212 and may be axially moveable relative to the collar. The lock 230 is configured to lock the implant 200 to the LAA and hold the LAA closed. The lock 230 may include an engagement member configured to move between a constrained delivery configuration and a radially expanded configuration, with the engagement member biased in the radially expanded configuration. The collar 212 may hold the engagement member in the constrained configuration, and axial movement of the shaft through the collar 212 moves the collar off the engagement member, allowing the engagement member to move into the radially expanded configuration.

In the embodiment shown in FIGS. 5-8, the engagement member of the lock 230 includes a plurality of arms 232, each arm 232 having a first end 234 coupled to the shaft adjacent the distal end 225 of the shaft 220. Each arm 232 extends proximally from the first end 234 to a second free end 236, the second free end configured to move between the constrained configuration and the radially expanded configuration. The collar 212 may hold the second free ends 236 against the shaft 220 adjacent the proximal end of the shaft in the constrained configuration, as shown in FIG. 5, and axial movement of the shaft within the collar releases the second free end of each of the plurality of arms, as will be discussed below.

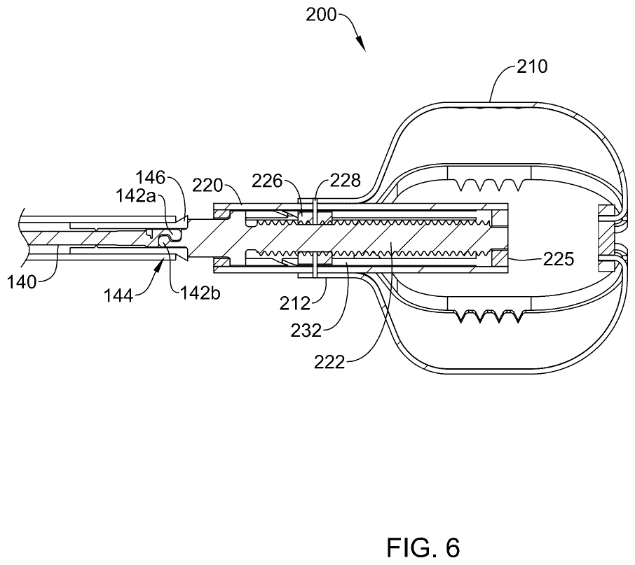

The cross-sectional view in FIG. 6 illustrates the elements of the delivery system, which may be the same as described above with reference to FIG. 2. The delivery system may include a delivery rod 140 with a first coupler 142a removably coupled to a second coupler 142b on the proximal end of the shaft 220, which together form a two-piece coupling arrangement 142a, 142b, such as a train latch. A removable cover 146 may be slidingly disposed over at least the two-piece coupling arrangement 142a, 142b and a portion of the delivery rod 140. The cover 146 may be fixed to the delivery sleeve 144, and the cover 146 may engage the rod 140 and first coupler 142a with a friction fit. The sleeve 144 and cover 146 may be disposed over the two-piece coupling arrangement 142a, 142b during delivery, allowing for rotation and axial movement of the sleeve 144 and rod 140 to be translated to rotation and axial movement of the occlusive implant 200. The two-piece coupling arrangement 142a, 142b may be configured to be released by lateral movement of the first coupler 142a relative to the second coupler 142b when the cover 146 has been withdrawn proximally, as discussed in more detail below. The covered coupling arrangement allows the rod 140 to rotate and drive the shaft 120 in both a counter-clockwise and a clockwise direction without becoming disengaged from the shaft.

The cross-sectional view in FIG. 6 also illustrates the elements of the shaft, including a fastener 222 and a nut 226 disposed around the fastener. The fastener 222 may be threaded and threadingly engaged with the nut 226. The nut 226 may have at least one opening in a sidewall thereof, and the collar 212 may have at least one aperture 218 through a sidewall thereof, where the opening in the nut 226 and the aperture in the collar 212 are aligned such that at least one pin 228 may extend through the aperture in the collar and the opening in the nut to secure the collar 212 to the nut 226. In the embodiment shown in FIG. 6, the nut 226 has two openings on opposite sides, the collar 212 has two apertures on opposite sides, and two pins 228 extend through the aligned openings and apertures to secure the collar to the nut. The fastener 222 threadingly engages the nut 226 such that rotation of the rod 240 in a first direction, such as counter clockwise, causes the fastener 222 to be withdrawn proximally through the nut 226 and collar 212, and rotation of the rod 240 in a second direction, such as clockwise, causes the fastener 222 to advance distally through the nut 226 and the collar 212 into the interior space defined by the expandable framework 210.

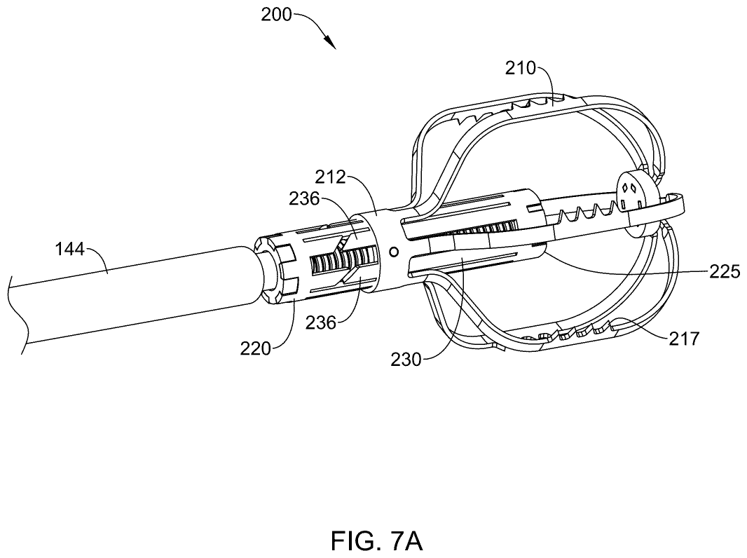

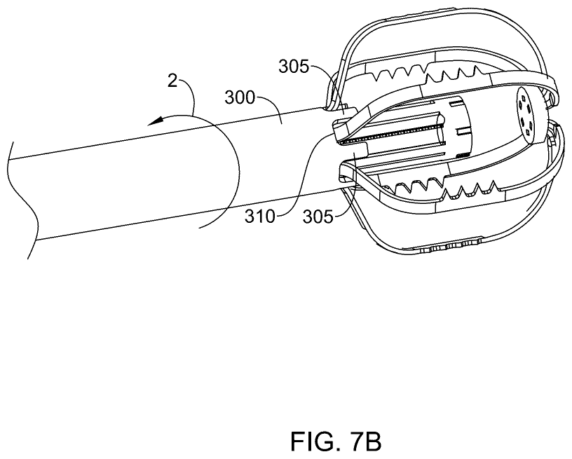

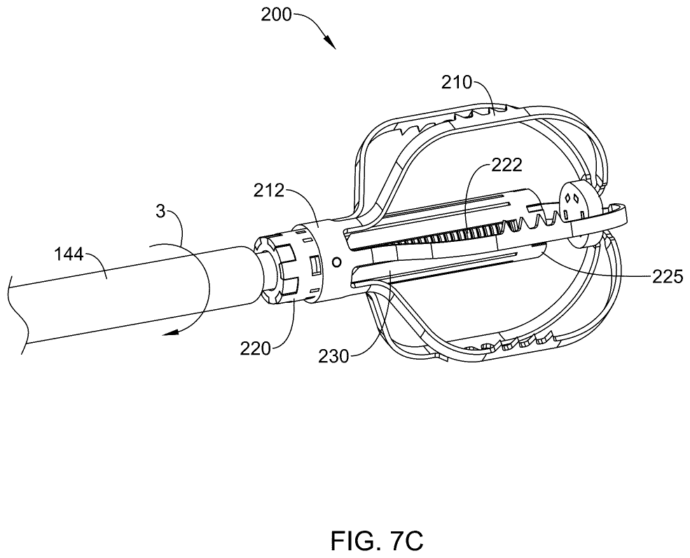

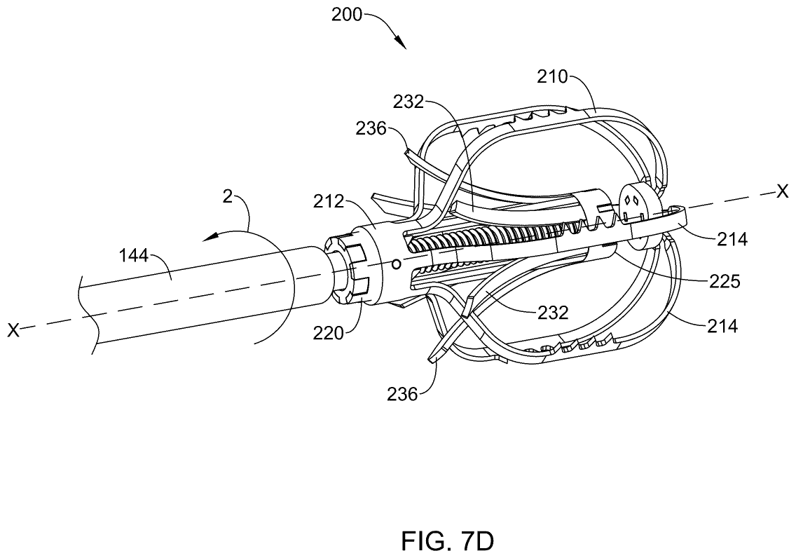

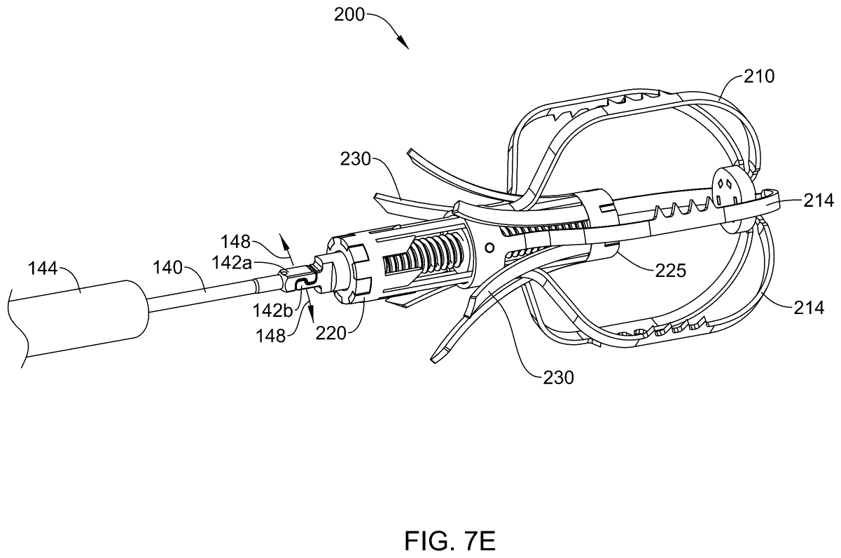

FIGS. 7A-7E illustrate the deployment of the lock arms 232. The implant 200 may be delivered into the LAA in a constrained configuration in a delivery sheath in which the struts 214 are substantially straight (not shown). When the delivery sheath is withdrawn proximally, the expandable framework 210 automatically expands radially into the configuration shown in FIG. 7A. In the delivery configuration and once the struts 214 have expanded, the collar 212 is positioned around a middle region of the shaft 220, with the collar 212 over the free ends 236 of the arms 232, holding the arms 232 in their axial, constrained configuration.

Once the implant 200 has been delivered to the LAA and the expandable framework 210 has radially expanded with at least the middle portion 213 of the struts 214 in contact with the inner walls of the LAA, a torque shaft 300 may be advanced over the rod 140 and shaft 220. As shown in FIG. 7B, the distal end of the torque shaft 300 may have a plurality of axially extending fingers 305 with recesses 310 between adjacent fingers, where the fingers 305 are configured to be positioned between the struts 214 and the recesses 310 engage the proximal ends of the struts 214. With the torque shaft 300 thus engaged with the expandable framework 210, rotation of the torque shaft 300 in the first direction, indicated by arrow 2, rotates the expandable framework 210 in the first direction, allowing the projections 217 on the expandable framework 210 to grip or become embedded in the inner wall of the LAA twisting the LAA around the implant 200 and closing off the opening of the LAA around the torque shaft 300. Once the implant 200 has been rotated and the LAA has twisted round the implant, the rod 140 and sleeve 144 may be rotated in the second direction opposite the first direction (clockwise in this example), indicated by arrow 3, which causes the shaft 220, including fastener 222, to move distally through the collar 212 (FIG. 7C). The torque shaft 300 may remain engaged with the struts 214 to hold the LAA twisted around the implant 200, however the torque shaft 300 is not rotated clockwise with the rod 140 and shaft 220. The torque shaft 300 may be held stationary while the rod 140 is rotated within it. The torque shaft 300 is not shown in FIGS. 7C-7E in order to illustrate the details of the shaft 220 and collar 212 during subsequent steps.

As the shaft 220 moves distally through the collar 212, the second free ends 236 of the plurality of arms move out from under the collar, allowing the arms to expand into the radially expanded configuration, as shown in FIG. 7D. As the collar 212 moves proximally off the arms 232, the second free end 236 of each arm bends radially away from a longitudinal axis of the shaft but remains facing proximally, as shown in FIG. 7D. When the shaft has moved distally into the interior space defined by the expandable framework 210, the collar 212 may be disposed over the proximal end of the shaft, as shown in FIG. 7D. As shown in the progression from FIG. 7C to FIG. 7D, the free end 236 of each arm swings through 30 to 60 degrees relative to the longitudinal axis X-X of the shaft 220. In the embodiment shown in the figures, each arm 232 moves through a space between adjacent struts 214.

Once the arms 232 have expanded, as shown in FIG. 7D, the rod 140 may be rotated in the first direction (counter-clockwise in this example), as shown by arrow 2, causing the fastener 222 to be withdrawn proximally through the nut 226 and collar 212, thereby moving the second free ends 236 of the arms proximally through the space between struts 214, as shown in the progression from FIG. 7D to FIG. 7E. Once the free ends 236 of the arms 232 have become implanted in the twisted tissue at what was the opening of the LAA, the torque shaft 300, the sleeve 144, and attached cover 146 may be withdrawn proximally relative to the rod 140, uncovering the coupling arrangement 142a, 142b, which is then configured to release the rod 140 from the proximal end of shaft 220. The rod 140 with its first coupler 142a may be moved laterally relative to the second coupler 142b, indicated by arrows 148, thereby uncoupling the first coupler 142a from the second coupler 142b, as shown in FIG. 7E. For example, the coupling arrangement 142a, 142b may be a train latch type structure.

The initial twisting of the expandable framework 210 within the LAA with the projections 217 embedded in the inner wall of the LAA may cause the tissue at the neck of the LAA to become twisted around the second coupler 142b such that once the rod and first coupler 142a are removed, the entirety of the implant 200 including the proximal end of the shaft 220 and the second coupler 142b, is located within the extravascular space or tissue of the LAA, and no structure of the implant system extends into the endovascular space within the heart.

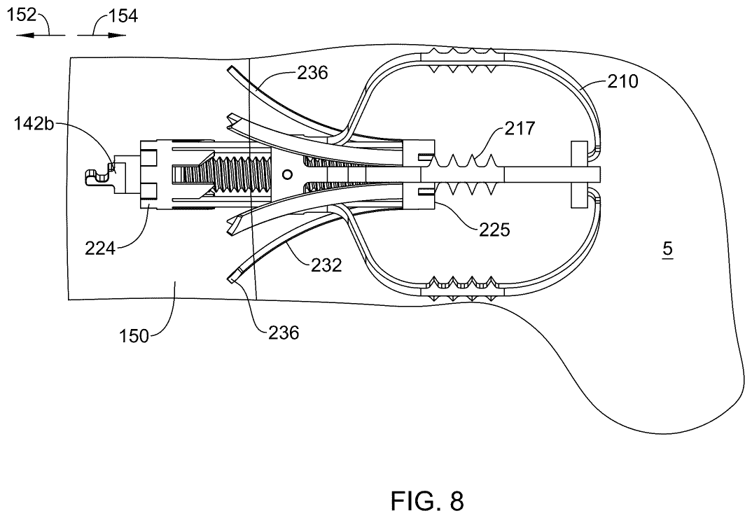

FIG. 8 shows the implant 200 deployed in the LAA 5. The projections 217 have engaged the inner wall of the LAA and the implant was rotated in the first direction 2 with the torque shaft 300, twisting the LAA and folding tissue at the neck of the LAA around the proximal end 224 of the shaft and the second coupler 142b such that the second coupler 142b is within the LAA or within the twisted tissue at the neck of the LAA. Rotation of the rod in the second direction 3 caused deployment of the arms 232 of the lock, followed by rotation of the rod in the first direction 2 to move the arms 232 proximally such that the second free ends 236 of the arms 232 become at least partially embedded in the tissue 150 at the neck of the LAA. The free ends 236 of arms 232 being embedded within the twisted tissue at the neck of the LAA may prevent the tissue from untwisting. The lock thus provides a secure closure without any structure in contact with the endovascular space 152 in the heart, eliminating device-related thrombus. In some embodiments, 0.01 inch to 0.2 inch of the second free ends 236 of the arms may be disposed within the tissue of the LAA. The shaft may have a length of 0.25 inch to 1.0 inch. Once the lock is deployed, the torque shaft 300 may be removed and the rod may be uncoupled from the shaft, leaving the entire implant 200 completely within the extravascular space 154 of the LAA, with no implant structure extending into the endovascular space 152 of the heart.

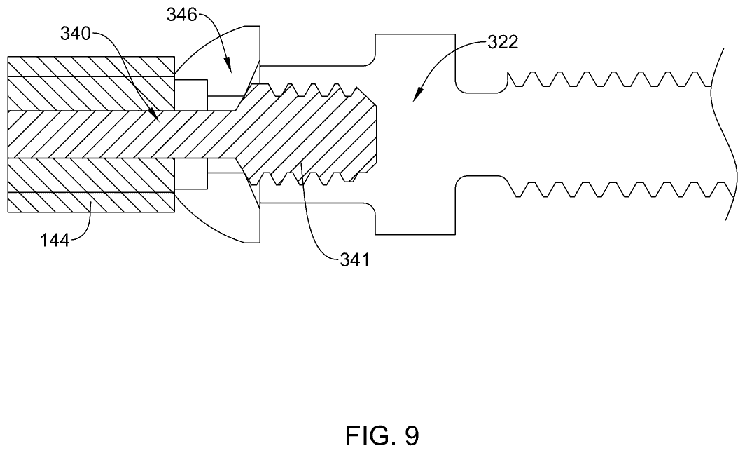

In other embodiments, instead of the two-piece coupling arrangement 142a, 142b, the implants 100, 200 described above may be threadingly coupled to the delivery rod. As shown in FIG. 9, a rod 340 with an externally threaded distal end 341 may be disposed within the delivery sleeve 144. A strain relief 346 may be disposed at the distal end of the delivery sleeve 144.

A fastener 322 with an internally threaded proximal end may be threaded onto the rod 340. The fastener 322 may be the same in all other features and structures as the fasteners 122, 222 described above. The threaded engagement between the rod 340 and fastener 322 allows for the rod to be rotated in a first direction to cause movement of the fastener relative to the nut and collar in a first direction, and when the rod is rotated in the second, opposite direction, the rod 340 disengages with the fastener 322 to leave the implant in the LAA.

The implants 100, 200 may both be used in a method of closing a left atrial appendage. The method includes inserting the implant into the LAA, where the implant includes an expandable framework having a proximal end coupled to a collar. The expandable framework is configured to expand from a collapsed delivery configuration to an expanded deployed configuration, and at least a portion of the expandable framework has a plurality of projections extending laterally therefrom. The implant also includes a shaft disposed within the collar and axially moveable relative to the collar, and a lock coupled to the shaft. The lock includes an engagement member configured to move between a constrained configuration and a radially expanded configuration, where when in the radially expanded configuration, the engagement member is configured to engage tissue at a proximal end of the shaft. The implant may be removably coupled to a delivery rod, through the proximal end of the shaft. After the implant has been inserted into the LAA, the method includes rotating the delivery rod in a first direction to rotate the shaft and expandable framework together in the first direction and engage the expandable framework projections with an inner surface of the left atrial appendage, followed by rotating the delivery rod in a second direction opposite the first direction to rotate the shaft in the second direction, thereby advancing the shaft distally through the collar to move the engagement member into the radially expanded configuration, wherein the engagement member engages tissue of the left atrial appendage at a proximal region of the shaft. At this point, the implant is secured within the LAA, and the delivery rod is removed, leaving the proximal end of the shaft completely within tissue of the left atrial appendage.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the occlusive implant, the delivery sheath, the core wire, the expandable framework, the occlusive element, the capsule, the elongate fingers, the elongate strand, etc. and/or elements or components thereof.

In some embodiments, the system and/or components thereof may be made from a metal, metal alloy, polymer, a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly (alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof, or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An implant for left atrial appendage closure, comprising:
an expandable framework having a proximal end coupled to a collar, the expandable framework configured to expand from a collapsed delivery configuration to an expanded deployed configuration;
a shaft disposed within the collar and axially moveable relative to the collar; and
a lock coupled to the shaft, the lock including an engagement member configured to move between a constrained configuration and a radially expanded configuration, wherein the engagement member is biased in the radially expanded configuration.

2. The implant of claim 1, wherein the collar holds the engagement member in the constrained configuration, and axial movement of the shaft within the collar moves the collar off the engagement member, allowing the engagement member to move into the radially expanded configuration.

3. The implant of claim 2, wherein the lock includes a plurality of arms, each arm having a first end coupled to the shaft and a second free end, the second free end configured to move between the constrained configuration and the radially expanded configuration.

4. The implant of claim 3, wherein the second free end of each arm extends in a proximal direction in the radially expanded configuration.

5. The implant of claim 3, wherein the collar holds the second free end of each arm in the constrained configuration, and axial movement of the shaft within the collar releases the second free end of each of the plurality of arms.

6. The implant of claim 1, wherein the expandable framework is biased in the expanded deployed configuration.

7. The implant of claim 1, wherein the expandable framework includes a plurality of struts each having a proximal end, a middle portion, and distal end, wherein the proximal ends are coupled to the collar, the distal ends are coupled together, and the middle portions are moveable between the collapsed delivery configuration and the expanded deployed configuration, wherein the plurality of struts are biased in the expanded deployed configuration.

8. The implant of claim 7, wherein at least the middle portion of each strut has a plurality of projections extending laterally from the strut.

9. The implant of claim 1, further comprising a delivery rod removably coupled to a proximal end of the shaft, the delivery rod configured to rotate the shaft and move the shaft axially relative to the expandable framework.

10. The implant of claim 9, wherein the shaft includes a fastener and a nut disposed around the fastener, the nut having at least one opening in a sidewall thereof, wherein the collar has at least one aperture through a sidewall thereof, the implant further comprising at least one pin extending through the aperture in the collar and the opening in the nut.

11. The implant of claim 10, wherein the fastener threadingly engages the nut such that rotation of the delivery rod in a first direction causes the fastener to move in a first axial direction through the nut and the collar, and rotation of the delivery rod in a second direction opposite the first direction causes the fastener to move in a second axial direction through the nut and the collar.

12. The implant of claim 3, wherein the first end of each of the plurality of arms is coupled adjacent a proximal end of the shaft, and each arm extends distally with the collar holding the second free end of each arm in the constrained configuration.

13. The implant of claim 12, wherein movement of the shaft distally through the collar moves the second free ends of the plurality of arms out from under the collar, allowing the arms to expand into the radially expanded configuration.

14. The implant of claim 13, wherein the second free end of each arm bends radially away from a longitudinal axis of the shaft, and then bends proximally as the shaft moves distally through the collar.

15. The implant of claim 3, wherein the first end of each of the plurality of arms is coupled adjacent a distal end of the shaft, and each arm extends proximally with the collar holding the second free end of each arm in the constrained configuration.

16. The implant of claim 15, wherein movement of the shaft distally through the collar moves the collar proximally off the second free ends of the plurality of arms, allowing the second free ends of the arms to bend radially away from a longitudinal axis of the shaft.

17. An implant for left atrial appendage closure, comprising:
an expandable framework having a proximal end coupled to a collar, the expandable framework configured to expand from a collapsed delivery configuration to a radially expanded configuration;
a shaft disposed within the collar and axially moveable relative to the collar; and
a lock coupled to the shaft, the lock including a plurality of arms each having a first end coupled to the shaft and a second free end configured to move between a constrained configuration when positioned under the collar and a radially expanded configuration when released from the collar, the plurality of arms biased in the radially expanded configuration;
wherein the shaft is configured to rotate in a first direction causing the shaft to move axially through the collar to move the plurality of arms into the radially expanded configuration.

18. The implant of claim 17, wherein the first end of each of the plurality of arms is coupled adjacent a proximal end of the shaft, and each arm extends distally with the collar holding the second free end of each arm in the constrained configuration.

19. The implant of claim 17, wherein the first end of each of the plurality of arms is coupled adjacent a distal end of the shaft, and each arm extends proximally with the collar holding the second free end of each arm in the constrained configuration.

20. A method of closing a left atrial appendage, comprising:
inserting an implant into the left atrial appendage, the implant including:
an expandable framework having a proximal end coupled to a collar, the expandable framework configured to expand from a collapsed delivery configuration to an expanded deployed configuration, at least a portion of the expandable framework having a plurality of projections extending laterally therefrom;
a shaft disposed within the collar and axially moveable relative to the collar;
a lock coupled to the shaft, the lock including an engagement member configured to move between a constrained configuration and a radially expanded configuration, wherein when in the radially expanded configuration, the engagement member is configured to engage tissue at a proximal end of the shaft; and
a delivery rod removably coupled to a proximal end of the shaft;
inserting a torque shaft over the delivery rod and into engagement with the expandable framework;
rotating the torque shaft in a first direction to rotate the expandable framework in the first direction and engage the plurality of projections with an inner surface of the left atrial appendage;
rotating the delivery rod relative to the torque shaft in a second direction opposite the first direction to rotate the shaft in the second direction, thereby axially moving the shaft through the collar to move the engagement member into the radially expanded configuration, wherein the engagement member engages tissue of the left atrial appendage at a proximal region of the shaft; and
removing the torque shaft and the delivery rod, leaving the proximal end of the shaft within tissue of the left atrial appendage.
